(19) United States
(12) Patent Application Publication
(10) Pub. No.: US 2018/0080071 A1
(43) Pub. Date: Mar. 22, 2018

(54) DETECTION OF NUCLEIC ACID MOLECULES USING NANOPORES AND COMPLEXING MOIETIES

(71) Applicant: Coyote Bioscience Co., Ltd., Beijing (CN)

(72) Inventors: Xiang Li, Beijing (CN); Kun Yang, Shijiazhuang City (CN)

(21) Appl. No.: 15/609,928

(22) Filed: May 31, 2017

Related U.S. Application Data
(63) Continuation of application No. PCT/CN2014/095909, filed on Dec. 31, 2014.

(51) Int. Cl. C12Q 1/68

(52) U.S. Cl. CPC C12Q 1/6869; C12Q 1/6806

(57) ABSTRACT

The present disclosure provides methods and systems for assaying the presence of a target nucleic acid molecule in a sample having or suspected of having the target nucleic acid molecule. A method of assaying the presence of the target nucleic acid molecule comprises facilitating the flow of the sample through at least one nanopore in a membrane disposed adjacent or in proximity to an electrode that is adapted to detect a current or change thereof upon movement of a complex having the target nucleic acid molecule coupled to the complexing moiety through the at least one nanopore. Next, the current or change thereof is measured with the electrode. The complex in the sample is detected from the current or change thereof, thereby assaying the presence of the target nucleic acid molecule in the sample.

### CROSS-REFERENCE

This application is a continuation of PCT International Application No. PCT/CN2014/095909, filed Dec. 31, 2014, which application is herein incorporated by reference in its entirety for all purposes.

### BACKGROUND

A nucleic acid molecule can be amplified, using, for example, thermal cycling based approaches (e.g., polymerase chain reaction (PCR)) or isothermal approaches (e.g., loop-mediated isothermal amplification). Concurrent with or subsequent to amplification of a nucleic acid molecule, amplified products can be detected. This can permit the identification of a nucleic acid sequence of interest such as single nucleotide polymorphisms (SNPs), sequence mutations (including e.g., deletions, insertions, duplication, and translocation), rare nucleic acid molecules/sequences, and other sequences of interest in a sample. Additionally, nucleic acid amplification may be used to prepare a nucleic acid molecule for nucleic acid sequencing.

### SUMMARY

Although there are methods and systems currently available for nucleic acid amplification and sequence identification, various limitations are associated with such methods. Some methods for the identification of a nucleic acid sequence are expensive and may not generate sequence information rapidly enough within a time frame and/or at an accuracy necessary for the intended application. Recognized herein is the need for improved methods for identifying products of nucleic acid amplification reactions, which may enable sequence identification.

The present disclosure provides systems and methods for readily assaying a presence or absence of a target nucleic acid sequence or molecule in a biological sample.

An aspect of the present disclosure provides a method for assaying the presence of a target nucleic acid molecule in a sample having or suspected of having the target nucleic acid molecule, the target nucleic acid molecule being coupled to a complexing moiety. The method comprises (a) facilitating the flow of the sample through at least one nanopore in a membrane disposed adjacent or in proximity to an electrode that is adapted to detect a current or change thereof upon movement of a complex having the target nucleic acid molecule coupled to the complexing moiety through the at least one nanopore, wherein the movement takes a dwell time that is longer than that of the movement of the target nucleic acid molecule through the at least one nanopore when the target nucleic acid molecule is not coupled to the complexing moiety; and (b) measuring the current or change thereof with the electrode upon facilitating the flow of the sample through the at least one nanopore; and (c) detecting the complex in the sample from the current or change thereof measured in (b) without obtaining a nucleic acid sequence of the target nucleic acid molecule, thereby assaying the presence of the target nucleic acid molecule in the sample.

Another aspect provides a method for assaying the presence of a target nucleic acid molecule in a sample having or suspected of having the target nucleic acid molecule, the target nucleic acid molecule being coupled to a protein other than a polymerase. The method comprises (a) facilitating the flow of the sample through at least one nanopore in a membrane disposed adjacent or in proximity to an electrode that is adapted to detect a current or change thereof upon movement of a complex having the target nucleic acid molecule coupled to the protein through the at least one nanopore, wherein the movement takes a dwell time that is longer than that of the movement of the target nucleic acid molecule through the at least one nanopore when the target nucleic acid molecule is not coupled to the protein; (b) measuring the current or change thereof with the electrode upon facilitating the flow of the sample through the at least one nanopore; and (c) detecting the complex in the sample from the current or change thereof measured in (b), thereby assaying the presence of the target nucleic acid molecule in the sample.

Another aspect provides a method for assaying the presence of a target nucleic acid molecule in a sample having or suspected of having the target nucleic acid molecule, the target nucleic acid molecule being coupled to an enzyme under conditions such that the enzyme is not enzymatically active. The method comprises (a) facilitating the flow of the sample through at least one nanopore in a membrane disposed adjacent or in proximity to an electrode that is adapted to detect a current or change thereof upon movement of a complex having the target nucleic acid molecule coupled to the enzyme through the at least one nanopore, wherein the movement takes a dwell time that is longer than that of the movement of the target nucleic acid molecule through the at least one nanopore when the target nucleic acid molecule is not coupled to the enzyme; (b) measuring the current or change thereof with the electrode upon facilitating the flow of the sample through the at least one nanopore; and (c) detecting the complex in the sample from the current or change thereof measured in (b), thereby assaying the presence of the target nucleic acid molecule in the sample. In some embodiments, the conditions are selected from the group consisting of salt concentration of the sample and temperature of the sample. In some embodiments, the salt concentration includes a concentration of Mg²⁺. In some embodiments, the concentration is less than 1 mole/liter (M). In some embodiments, the concentration is less than 0.1 M. In some embodiments, the concentration is less than 0.01 M. In some embodiments, the concentration is less than 0.001 M.

In some embodiments, the complexing moiety, protein or enzyme is coupled to the membrane. In some embodiments, the complexing moiety, protein or enzyme is covalently coupled to the membrane.

In some embodiments, the complexing moiety, protein or enzyme is coupled to the nanopore. In some embodiments, the complexing moiety, protein or enzyme is covalently coupled to the nanopore.

In some embodiments, the complexing moiety is a protein. In some embodiments, the protein is an endonuclease or exonuclease. In some embodiments, the protein binds to the target nucleic acid molecule at a binding strength that is greater than a binding strength for any other nucleic acid molecule.

In some embodiments, the complexing moiety is a primer. In some embodiments, the primer is a universal primer.

In some embodiments, the sample includes the complex. In some embodiments, the sample has a Mg²⁺ concentration that is less than 1 M. In some embodiments, the concentration is less than 0.1 M. In some embodiments, the concentration is less than 0.01 M. In some embodiments, the concentration is less than 0.001 M.

In some embodiments, the complexing moiety, protein or enzyme reversibly couples to the target nucleic acid molecule. In some embodiments, the complexing moiety, protein or enzyme is removable from the target nucleic acid molecule upon the application of an electric field and/or pressure pulse.

In some embodiments, prior to the step of (a) referenced above, the steps of (i) a reaction mixture is provided that includes a biological sample having or suspected of having a template nucleic acid molecule as a precursor of the target nucleic acid molecule, at least one primer that is complementary to the template nucleic acid molecule, and a polymerase, and (ii) the reaction mixture is subjected to a nucleic acid amplification reaction under conditions that yield the target nucleic acid molecule in the sample. In some embodiments, the complexing moiety, protein or enzyme is provided during or subsequent to the nucleic acid amplification reaction. In some embodiments, the sample comprises the target nucleic acid molecule. In some embodiments, the target nucleic acid molecule is a copy among multiple copies as amplification products of the nucleic acid amplification reaction. In some embodiments, the primer has one or more restriction sites or binding sites for the complexing moiety, protein or enzyme. In some embodiments, the nucleic acid amplification reaction is polymerase chain reaction (PCR). In some embodiments, the nucleic acid amplification reaction is isothermal amplification. In some embodiments, the isothermal amplification is loop mediated isothermal amplification (LAMP). In some embodiments, the at least one primer includes at least two primers.

In some embodiments, the complexing moiety, protein or enzyme specifically binds to the target nucleic acid molecule. In some embodiments, the step of (b) referenced above comprises measuring a change in current, which change is indicative of the presence of the complex. In some embodiments, the change in current is a first moment of current with time.

In some embodiments, the current is measured subsequent to facilitating the flow of the sample through the at least one nanopore. In some embodiments, the complexing moiety, protein or enzyme reversibly couples to the target nucleic acid molecule. In some embodiments, the at least one nanopore has a cross-sectional size that is from about 0.5 nanometer (nm) to 30 nm. In some embodiments, the cross-sectional size is from about 1 nm to 20 nm. In some embodiments, the cross-sectional size is from about 2.5 nm to 3.4 nm.

In some embodiments, the membrane is a lipid bilayer. In some embodiments, the membrane is a solid state membrane. In some embodiments, the solid state membrane includes a semiconductor or non-metal. In some embodiments, the solid state membrane includes a material selected from the group consisting of carbon, silicon, germanium and gallium arsenide. In some embodiments, the solid state membrane is formed of graphene.

In some embodiments, the at least one nanopore is a pore-forming protein in the membrane. In some embodiments, the pore-forming protein is alpha hemolysin or MspA porin.

In some embodiments, the facilitating comprises applying an electrical potential across the at least one nanopore. In some embodiments, the electrical potential is reversible. In some embodiments, the electrical potential is from about 1 V to 10 V relative to a reference.

In some embodiments, the method further comprises applying a pulse of a pressure drop or an electrical potential across the at least one nanopore to decouple the complexing moiety, protein or enzyme from the target nucleic acid molecule, which pulse is applied subsequent to facilitating the flow of the sample through the at least one nanopore. In some embodiments, the at least one nanopore is adjacent or in proximity to an additional electrode. In some embodiments, the target nucleic acid molecule is detected by (i) measuring the current or change thereof upon the flow of the sample through at least one nanopore and (ii) comparing the current or change thereof to a reference. In some embodiments, the complexing moiety, protein or enzyme increases the dwell time upon interaction of the complexing moiety, protein or enzyme with the at least one nanopore.

In some embodiments, the at least one nanopore includes a plurality of nanopores. In some embodiments, the plurality of nanopores is individually addressable. In some embodiments, the target nucleic acid molecule is detected without obtaining a nucleic acid sequence of the target nucleic acid molecule from sequential measurements of the current or change thereof upon the flow of the sample through the at least one nanopore. In some embodiments, the current or change thereof is detected at a dwell time that is indicative of the presence of the target nucleic acid molecule. In some embodiments, the target nucleic acid molecule includes at least 5 contiguous nucleotide bases. In some embodiments, the target nucleic acid molecule includes at least 10 contiguous nucleotide bases. In some embodiments, the target nucleic acid molecule includes at least 20 contiguous nucleotide bases. In some embodiments, the target nucleic acid molecule is single stranded. In some embodiments, the target nucleic acid molecule is double stranded. In some embodiments, the target nucleic acid molecule is deoxyribonucleic acid (DNA) or ribonucleic acid (RNA).

Another aspect of the present disclosure provides a system for assaying the presence of a target nucleic acid molecule in a sample having or suspected of having the target nucleic acid molecule, the target nucleic acid molecule including at least 5 contiguous nucleotide bases. The system comprises at least one nanopore in a membrane that is disposed adjacent or in proximity to an electrode, wherein the electrode is adapted to detect a current upon flow of a sample through the at least one nanopore; at least one sample holder in fluid communication with the at least one nanopore and adapted to retain the sample; and a computer processor that is operatively coupled to the electrode and programmed to (i) facilitate the flow of the sample from the at least one sample holder through the at least one nanopore, (ii) measure a dwell time of an individual nucleic acid molecule in or through the nanopore, and (iii) identify the individual nucleic acid molecule as the target nucleic acid molecule when the dwell time falls within a reference threshold.

In some embodiments, the computer processor is programmed to measure a first dwell time of the individual nucleic acid molecule through the nanopore and identify the individual nucleic acid molecule as the target nucleic acid molecule if the first dwell is longer than a second dwell time of the target nucleic acid molecule in or through the at least one nanopore when the target nucleic acid molecule is not coupled to a complexing moiety.

In some embodiments, the complexing moiety is a protein. In some embodiments, the protein is not a polymerase. In some embodiments, the protein is an endonuclease or exonuclease.

In some embodiments, the complexing moiety is a primer. In some embodiments, the primer is a universal primer.

In some embodiments, the target nucleic acid molecule includes at least 10 contiguous nucleotide bases. In some embodiments, the target nucleic acid molecule includes at least 20 contiguous nucleotide bases. In some embodiments, the target nucleic acid molecule is single stranded. In some embodiments, the target nucleic acid molecule is double stranded.

In some embodiments, the computer processor is programmed to identify the individual nucleic acid molecule as at least a portion of the target nucleic molecule without obtaining a nucleic acid sequence of the individual nucleic acid molecule.

In some embodiments, the sample has a Mg²⁺ concentration that is less than 1 M. In some embodiments, the concentration is less than 0.1 M. In some embodiments, the concentration is less than 0.01 M. In some embodiments, the concentration is less than 0.001 M.

In some embodiments, the computer processor is programmed to (a) measure a current or change thereof, and (b) determine the dwell time from the current or change thereof. In some embodiments, the current or change thereof is measured relative to a baseline. In some embodiments, the computer processor is programmed to measure the current or change thereof subsequent to facilitating the flow of the sample through the at least one nanopore. In some embodiments, the computer processor is programmed to determine the dwell time upon comparison of the current or change thereof to a reference.

In some embodiments, the at least one nanopore has a cross-sectional size that is from about 0.5 nanometer (nm) to 30 nm. In some embodiments, the cross-sectional size is from about 1 nm to 20 nm. In some embodiments, the cross-sectional size is from about 2.5 nm to 3.4 nm.

In some embodiments, the membrane is a lipid bilayer. In some embodiments, the membrane is a solid state membrane. In some embodiments, the solid state membrane includes a semiconductor or non-metal. In some embodiments, the solid state membrane includes a material selected from the group consisting of carbon, silicon, germanium and gallium arsenide. In some embodiments, the solid state membrane is formed of graphene.

In some embodiments, the at least one nanopore is a pore-forming protein in the membrane. In some embodiments, the pore-forming protein is alpha hemolysin or MspA porin.

In some embodiments, the computer processor is programmed to apply an electrical potential across the nanopore. In some embodiments, the electrical potential is reversible. In some embodiments, the electrical potential is from about 1 V to 10 V relative to ground. In some embodiments, the computer processor is programed to apply a pulse of an electrical potential across the nanopore, wherein the pulse decouples a complexing moiety coupled to the target nucleic acid molecule.

In some embodiments, the nanopore is adjacent or in proximity to an additional electrode. In some embodiments, the additional electrode is a reference electrode. In some embodiments, the at least one nanopore includes a plurality of nanopores. In some embodiments, the plurality of nanopores is individually addressable.

In some embodiments, the at last one nanopore is part of a chip. In some embodiments, n the computer processor is separate from the chip. In some embodiments, the computer processor is part of a mobile electronic device.

In some embodiments, the computer processor is part of a circuit having the electrode. In some embodiments, the computer processor is separate from a circuit having the electrode. In some embodiments, the computer processor is an application specific integrated circuit (ASIC).

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

The novel features of the invention are set forth with particularity in the appended claims. A better understanding of the features and advantages of the present invention will be obtained by reference to the following detailed description that sets forth illustrative embodiments, in which the principles of the invention are utilized, and the accompanying drawings (also “figure” and “FIG.” herein), of which:

FIG. 1 shows a general workflow for the detection of a target nucleic acid molecule.

FIG. 2 shows a nanopore sensor comprising a membrane with a nanopore.

FIG. 3A shows a complexing moiety coupled to a membrane having a nanopore of a nanopore sensor, and a target nucleic acid molecule adjacent to the membrane; FIG. 3B shows the target nucleic acid threaded through the nanopore and coupled to the complexing moiety of FIG. 3A.

FIG. 4 shows a plot of current (i) with time measured by a nanopore sensor.

FIG. 5 shows a computer control system that is programmed or otherwise configured to implement methods provided herein.

### DETAILED DESCRIPTION

While various embodiments of the invention have been shown and described herein, it will be obvious to those skilled in the art that such embodiments are provided by way of example only. Numerous variations, changes, and substitutions may occur to those skilled in the art without departing from the invention. It should be understood that various alternatives to the embodiments of the invention described herein may be employed.

The term “membrane,” as used herein, generally refers to a structure that separates at least two volumes of a fluid. Examples of membranes include without limitation solid state membranes and lipid bilayers. A membrane may be an organic membrane, such as a lipid bilayer, or a synthetic membrane, such as a membrane formed of a solid state material (e.g., semiconductor, metal, semi-metal or non-metal) or polymeric material.

The term “nanopore,” as used herein, generally refers to a pore, channel or passage formed or otherwise provided in a membrane. The nanopore may be disposed adjacent or in proximity to a sensing circuit or an electrode coupled to a sensing circuit, such as, for example, a complementary metal-oxide semiconductor (CMOS) or field effect transistor (FET) circuit. In some examples, a nanopore has a characteristic size (e.g., cross-section, width or diameter) on the order of 0.1 nanometers (nm) to about 1000 nm. Some nanopores are proteins. Alpha hemolysin is an example of a protein nanopore.

The term “nucleic acid,” as used herein, generally refers to a molecule comprising one or more nucleic acid subunits. A nucleic acid may include one or more subunits selected from adenosine (A), cytosine (C), guanine (G), thymine (T) and uracil (U), or variants thereof. A nucleotide can include A, C, G, T or U, or variants thereof including but not limited to peptide nucleic acid (PNA). A nucleotide can include any subunit that can be incorporated into a growing nucleic acid strand. Such subunit can be an A, C, G, T, or U, or any other subunit that is specific to one or more complementary A, C, G, T or U, or complementary to a purine (i.e., A or G, or variant thereof) or a pyrimidine (i.e., C, T or U, or variant thereof). A subunit can enable individual nucleic acid bases or groups of bases (e.g., AA, TA, AT, GC, CG, CT, TC, GT, TG, AC, CA, or uracil-counterparts thereof) to be resolved. In some examples, a nucleic acid is deoxyribonucleic acid (DNA) or ribonucleic acid (RNA), or derivatives thereof. A nucleic acid may be single-stranded or double stranded. A nucleic acid may comprise one or more modified nucleotides, such as methylated nucleotides and nucleotide analogs.

The term “polymerase,” as used herein, generally refers to any enzyme capable of catalyzing a polymerization reaction. Examples of polymerases include, without limitation, a nucleic acid polymerase, a transcriptase or a ligase. A polymerase can be a polymerization enzyme.

The term “complexing moiety,” as used herein, generally refers to any atomic or molecular species that couples (e.g., attaches) with a nucleic acid molecule. A complexing moiety can be a protein, such as an enzyme. The complexing moiety can have an active site that interacts with a given target nucleic acid molecule. The interaction can be reversible or irreversible.

The term “subject,” as used herein, generally refers to an animal or other organism, such as a mammalian species (e.g., human), avian (e.g., bird) species, or plant. Mammals include, but are not limited to, murines, simians, humans, farm animals, sport animals, and pets. A subject can be an individual that has or is suspected of having a disease or a pre-disposition to the disease, or an individual that is in need of therapy or suspected of needing therapy. A subject can be a patient.

The term “sample,” as used herein, generally refers to any sample containing or suspected of containing a nucleic acid molecule. For example, a subject sample can be a biological sample containing one or more nucleic acid molecules. The biological sample can be obtained (e.g., extracted or isolated) from a bodily sample of a subject that can be selected from blood (e.g., whole blood), plasma, serum, urine, saliva, mucosal excretions, sputum, stool and tears. The bodily sample can be a fluid or tissue sample (e.g., skin sample) of the subject. In some examples, the sample is obtained from a cell-free bodily fluid of the subject, such as whole blood. In such instance, the sample can include cell-free DNA and/or cell-free RNA. In some other examples, the sample is an environmental sample (e.g., soil, waste, ambient air and etc.), industrial sample (e.g., samples from any industrial processes), and food samples (e.g., dairy products, vegetable products, and meat products).

The term “genome variation,” as used herein, generally refers to a variant or polymorphism in a nucleic acid sample or genome of a subject. Examples of variants include single nucleotide polymorphism, single nucleotide variant, insertion, deletion, substitution, repeat, variable length tandem repeat, flanking sequence, structural variant, transversion, rearrangement and copy number variation.

### Assaying the Presence of a Target Nucleic Acid Molecule

An aspect of the present disclosure provides methods and systems for assaying the presence of a target nucleic acid molecule in a sample. The target nucleic acid molecule can have a nucleic acid sequence of interest for an intended application, including without limitation, species identification, environmental testing, forensic analysis, and general research and disease characterization.

A sensor can be used to detect the presence of the target nucleic acid molecule in the sample. The sensor can have an array of one or more nanopores that are configured to detect current or a change in current with time. The target nucleic acid molecule can be detected by measuring current (C) or current change (or first moment of current with time, dC/dt) with time, and in some cases comparing such measurement to a reference (or baseline).

The sample can include one or more molecules, at least some of which can be the target nucleic molecule. A dwell time (or residence time) of any molecule in or through the nanopore can be indicative of the presence of the target nucleic molecule in the sample. In some situations, the target nucleic acid molecule has a detectable dwell time in the nanopore, which can be greater than other molecules in the sample. By measuring current or current change with time and determining dwell times, the target nucleic acid molecule, if present, can be detected in the sample.

The dwell time of the target nucleic acid molecule can be increased using a complexing moiety that couples (e.g., attaches) to the target nucleic acid molecule to form a complex that slows the flow of the target nucleic acid molecule through the nanopore. The complexing moiety can be a protein, such as an enzyme having an active site that couples to a portion of the target nucleic acid molecule. The complexing moiety can increase the dwell time upon interaction of the complex with the nanopore or membrane, or upon interaction of the complexing moiety with the target nucleic molecule.

The coupling of the complexing moiety to the target nucleic acid molecule can be reversible such that, upon the application of a stimulus, the coupling between the target nucleic acid molecule and the complexing moiety can be removed. Such stimulus can be a voltage, such as a voltage pulse (e.g., a 10V pulse).

The target nucleic acid molecule can be deoxyribonucleic acid (DNA), ribonucleic acid (RNA), or a variant thereof. The target nucleic acid sample can be processed, such as by fragmenting the target nucleic acid sample into fragments. The target nucleic acid molecule can be single stranded or double stranded.

The target nucleic acid molecule can include contiguous nucleotides. In some examples, the target nucleic acid molecule includes at least 5, 10, 30, 40, 50, 100, 200, 300, 400, 500, or 1000 nucleotides.

The target nucleic acid molecule can be an amplification product of a template nucleic acid molecule in the sample. In some cases, the target nucleic acid molecule can be detected by obtaining a biological sample from a subject and subjecting the sample to nucleic acid amplification to amplify at least a portion of the template nucleic acid molecule. Nucleic acid amplification can be performed under conditions that are selected to amplify the template nucleic acid molecule or a portion thereof if a nucleic acid sequence of interest is present. If the nucleic acid sequence of interest is present, nucleic acid amplification can yield one or more amplified nucleic acid products. Such products can include the target nucleic acid molecule.

The template nucleic acid molecule can be DNA, RNA, or a variant thereof. The template nucleic acid sample can be processed, such as by fragmenting the template nucleic acid sample into fragments. The template nucleic acid molecule can be single stranded or double stranded.

Once the sample has been subjected to nucleic acid amplification, the target nucleic acid molecule can be detected. This can be performed using sensors described elsewhere herein. The target nucleic acid molecule can be detected without nucleic acid sequencing, such as obtaining a nucleic acid sequence of the target nucleic acid molecule or other nucleic acid molecule in the sample. For example, the presence of the target nucleic acid molecule can be determined without sequencing by synthesis techniques (e.g., Illumina, Pacific Biosciences of California, Genia or Ion Torrent). The presence of the target nucleic acid molecule can be determined without sequential measurements of a signal(s) (e.g., an optical signal or current) that is indicative of at least 1, 2, 3, 4 or 5 nucleotides of the target nucleic acid molecule.

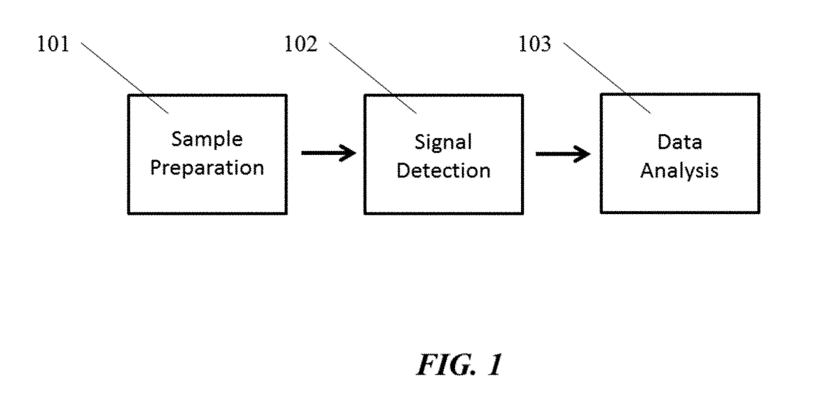

FIG. 1 shows a workflow for sample processing. In a first operation 101, a biological sample is prepared for detection. The biological sample can be obtained from a bodily fluid of a subject, for example, and a nucleic acid molecule can be isolated from the bodily fluid. The nucleic acid molecule can be a template nucleic acid molecule for subsequent analysis. In some cases, the nucleic acid molecule is processed to yield the template nucleic acid molecule, such as fragmented to yield multiple template nucleic acid molecules.

The template nucleic acid molecule can be subsequently subjected to nucleic acid amplification conditions to amplify (i.e., generate one or more copies) the template nucleic acid molecule. An amplification product of the template nucleic acid molecule can be a target nucleic acid molecule for subsequent analysis.

In some cases, a reaction mixture comprising a biological sample having or suspected of having the template nucleic acid molecule as a precursor of the target nucleic acid molecule is provided. The reaction mixture can also include at least one primer that is complementary to the template nucleic acid molecule and a polymerase. The at least one primer can include 1, 2, 3, 4, 5, 6, 7, 8, 9, 10, 20, 30, 40, or 50 primers. Each primer can have a sequence that is selected for a particular type of analysis, such as detecting a given disease or genome variation in a subject. The primer can have one or more restriction sites or binding sites for a complexing moiety.

Next, the reaction mixture can be subjected to a nucleic acid amplification reaction under conditions that yield the target nucleic acid molecule in the sample. The target nucleic acid molecule can be a copy among multiple copies of the template nucleic acid molecule, which are amplification products of the nucleic acid amplification reaction.

The reaction mixture can include reagents necessary to complete nucleic acid amplification (e.g., DNA amplification, RNA amplification), with non-limiting examples of such reagents including primer sets having specificity for target RNA or target DNA, DNA produced from reverse transcription of RNA, a DNA polymerase, a reverse transcriptase (e.g., for reverse transcription of RNA), suitable buffers (including zwitterionic buffers), co-factors (e.g., divalent and monovalent cations), dNTPs, and other enzymes (e.g., uracil-DNA glycosylase (UNG)), etc). In some cases, reaction mixtures can also comprise one or more reporter agents. The reaction mixture can also include an enzyme that is suitable to facilitate nucleic acid amplification, such as a polymerizing enzyme (also “polymerase” herein). The polymerase can be a DNA polymerase for amplifying DNA. Any suitable DNA polymerase may be used, including commercially available DNA polymerases. The DNA polymerase can be capable of incorporating nucleotides to a strand of DNA in a template bound fashion. Non-limiting examples of DNA polymerases include Taq polymerase, Tth polymerase, Tli polymerase, Pfu polymerase, VENT polymerase, DEEPVENT polymerase, EX-Taq polymerase, LA-Taq polymerase, Expand polymerases, Sso polymerase, Poc polymerase, Pab polymerase, Mth polymerase, Pho polymerase, ES4 polymerase, Tru polymerase, Tac polymerase, Tne polymerase, Tma polymerase, Tih polymerase, Tfi polymerase, Platinum Taq polymerases, Hi-Fi polymerase, Tbr polymerase, Tfl polymerase, Pfutubo polymerase, Pyrobest polymerase, Pwo polymerase, KOD polymerase, Bst polymerase, Sac polymerase, Klenow fragment, and variants, modified products and derivatives thereof. For certain Hot Start Polymerase, a denaturation step at 94° C. -95° C. for 2 minutes to 10 minutes may be required, which may change the thermal profile based on different polymerases.

In some cases, a DNA sample can be generated from an RNA sample. This can be achieved using reverse transcriptase, which can include an enzyme that is capable of incorporating nucleotides to a strand of DNA, when bound to an RNA template. Any suitable reverse transcriptase may be used. Non-limiting examples of reverse transcriptases include HIV-1 reverse transcriptase, M-MLV reverse transcriptase, AMV reverse transcriptase, telomerase reverse transcriptase, and variants, modified products and derivatives thereof.

Nucleic acid amplification reaction can include one or more primer extension reactions to generate amplified product(s). In PCR, for example, a primer extension reaction can include a cycle of incubating a reaction mixture at a denaturation temperature for a denaturation duration and incubating a reaction mixture at an elongation temperature for an elongation duration. Denaturation temperatures may vary depending upon, for example, the particular biological sample analyzed, the particular source of target nucleic acid (e.g., viral particle, bacteria) in the biological sample, the reagents used, and/or the desired reaction conditions. For example, a denaturation temperature may be from about 80° C. to about 110° C. In some examples, a denaturation temperature may be from about 90° C. to about 100° C. In some examples, a denaturation temperature may be from about 90° C. to about 97° C. In some examples, a denaturation temperature may be from about 92° C. to about 95° C. In still other examples, a denaturation temperature may be at least about 80°, 81° C., 82° C., 83° C., 84° C., 85° C., 86° C., 87° C., 88° C., 89° C., 90° C., 91° C., 92° C., 93° C., 94° C., 95° C., 96° C., 97° C., 98° C., 99° C., or 100° C.

As an alternative, in isothermal amplification, the temperature can be fixed (i.e., not cycled), and amplification product(s) can be generated using a primer set and a polymerase with high strand displacement activity in addition to a replication activity. An example of a polymerase that may be suitable for use in isothermal amplification is Bst polymerase. The temperature can be fixed between about 50° C. and 80° C., or 60° C. and 65° C. In loop mediated isothermal amplification (LAMP), for example, a template nucleic acid molecule can be amplified using a polymerase and a primer set having at least 2, 3, 4, or 5 primers.

During or subsequent to nucleic acid amplification, a complexing moiety can be provided to the reaction mixture. The complexing moiety can permit detection of the target nucleic acid molecule using a nanopore sensor of the present disclosure.

With continued reference to FIG. 1, in a second operation 102, subsequent to subjecting the template nucleic acid molecule to nucleic acid amplification, the presence of the target nucleic molecule as an amplification product can be determined. This can be achieved by detecting one or more signals that are indicative of the presence of the target nucleic acid molecule, such as dwell time of the target nucleic acid molecule upon measurements of current or a change in current with time using sensors described elsewhere herein. Next, in a third operation 103, the one or more signals are analyzed to determine whether the target nucleic acid molecule is present or not present. The one or more signals can also be analyzed to determine a relative quantity of the target nucleic molecule.

The amplification of the template nucleic acid molecule and detection of the target nucleic acid molecule can be performed in the same system, such as vessel. In some cases, the system is a tube that is configured for nucleic acid amplification, such as an eppendorf PCR tube. As an alternative, amplification and detection are in separate systems. For example, amplification is performed in an eppendorf PCR tube and detection is performed in a separate chip having a nanopore sensor.

### Nanopore Sensors

Another aspect of the present disclosure provides nanopore sensors for detecting a target nucleic acid molecule. A nanopore sensor can include an array of one or more nanopores in a membrane. Each nanopore can be disposed adjacent to a measurement electrode that is configured to detect a current or current change with time, in some cases with reference to a reference electrode.

The array can include at least 1, 2, 3, 4, 5, 6, 7, 8, 9, 10, 100, 200, 300, 400, 500, 1000, 10000, 100000 or 1000000 sensors. Each sensor can include at least 1, 2, 3, 4 or 5 nanopores. Each sensor can be individually addressable. The density of nanopores can be at least about 100, 200, 300, 400, 500, 600, 700, 800, 900, 1000, 10000, 100000, 10⁶, 10⁷, 10⁸, 10⁹, 10¹⁰, or 10¹¹ nanopores per square millimeter (mm²).

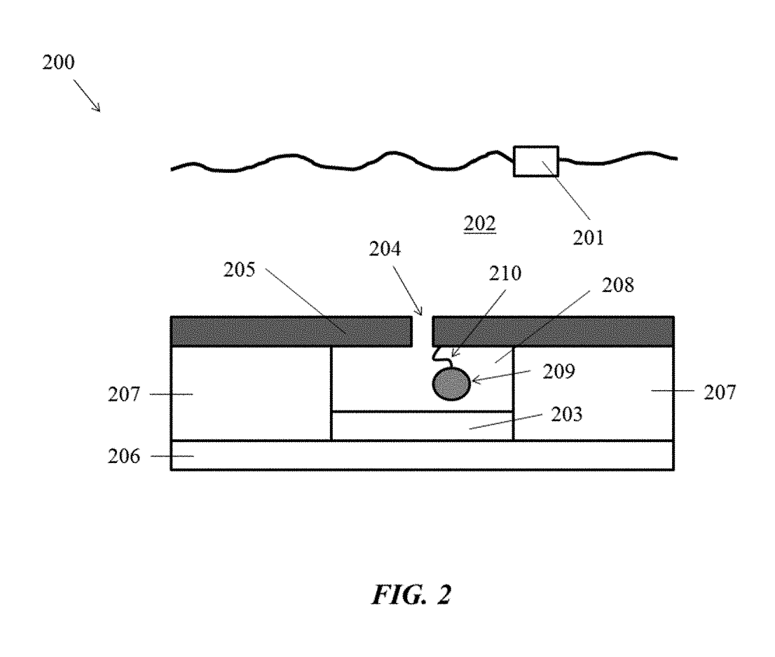

FIG. 2 shows a nanopore sensor 200 comprising a first electrode 201 in contact with a conductive solution 202 (e.g., salt solution). The sensor 200 comprises a second electrode 203 near, adjacent, or in proximity to a nanopore 204 in a membrane 205. The second electrode 203 is adjacent to a circuit element 206 having electrical circuitry for signal (e.g., current or current change) measurements. The membrane 205 is adjacent to a chamber 208 (e.g., well) that is at least partially defined by a wall 207. The wall 207 can be formed of a semiconductor, such as silicon oxide or aluminum oxide (e.g., SiO₂). As an alternative, the wall 207 is formed of a polymeric material. In some examples, the wall 207 is part of a tube that is usable for nucleic acid amplification.

The nanopore sensor 200 can be in a container (e.g., tube) that is configured for nucleic acid amplification, such as an eppendorf PCR tube. The container can include a top chamber for nucleic acid amplification of a template nucleic acid molecule and a bottom chamber for subsequent detection of the target nucleic acid molecule. The container can be disposable and/or reusable.

As an alternative, the nanopore sensor 200 can be part of a chip that includes a sample holder. The sample holder can contain a sample having or suspected of having a target nucleic acid molecule. The chip can have onboard electronics (e.g., a computer processor) for signal detection and processing. As an alternative, the onboard electronics can be off-chip, such as in a computer system adjacent to the chip and in communication with the chip. The chip can be disposable and/or reusable. The circuit element 206 can include electrical current flow paths that bring the nanopore sensor 200 in communication with the computer system.

For example, the nanopore sensor 200 is part of a container or chip that is insertable into and removable from a reader (not shown). The reader can include a computer processor that permits the detection of the target nucleic acid molecule in a sample having or suspected of having the target nucleic acid molecule. As alternative, the computer processor is in a computer system that is separate from and in communication with the reader. The reader can include a fluid flow system (e.g., pumps and actuators) that directs the sample to the nanopore sensor.

The nanopore sensor 200 can include an array of at least 1, 2, 3, 4, 5, 6, 7, 8, 9, 10, 100, 200, 300, 400, 500, 1000, 10000, 100000 or 1000000 sensors. Each sensor can include at least 1, 2, 3, 4 or 5 nanopores.

The membrane 205 can be a solid state membrane. The membrane 205 can be formed of a semiconductor or non-metal. In some examples, the membrane 205 is formed of a material selected from carbon, silicon, germanium and gallium arsenide. For example, the membrane 205 can be formed of graphene.

As an alternative, the membrane 205 can be a lipid bilayer. The lipid bilayer can include two layers of lipid molecules. The lipid bilayer can include phospholipids with hydrophilic head and two hydrophobic tails each. When exposed to water, such phospholipids can arrange themselves into a two-layered sheet (a bilayer) with all of their tails pointing toward the center of the sheet. The center of this bilayer can contain little to no water and exclude molecules. An outer surface of the lipid bilayer can be hydrophilic while an inner portion of the lipid bilayer can be hydrophobic.

The nanopore 204 can be a hole providing a channel through the membrane 205. As an alternative, the nanopore 204 can be a pore-forming protein in the membrane 205. Such alternative can be used in situations in which the membrane 205 is a lipid bilayer. The pore-forming protein can be alpha hemolysin or MspA porin.

The nanopore 204 can have a cross-sectional size that permits fluid flow through the nanopore 204. The cross-sectional size can permit flow of a nucleic acid sample through the nanopore 204. The cross-sectional size can be from about 0.5 nanometer (nm) to 30 nm, or 1 nm to 20 nm, 2 nm to 15 nm, 3 nm to 10 nm, or 2.5 nm to 3.4 nm.

The nanopore 204 can have various shapes and sizes. For example, the nanopore 204 can have a rectangular shape, hour glass shape, concave shape, convex shape, a conical shape, or partial shapes or combinations thereof. The nanopore 204 can have a length that spans the membrane 205. In some cases, the nanopore 204 has a length from about 10 nm to 5000 nm, or 20 nm to 1000 nm, or 30 nm to 1000 nm, and the membrane 205 has a thickness from about 10 nm to 5000 nm, or 20 nm to 1000 nm, or 30 nm to 1000 nm. The length of the nanopore 204 can be the same as the thickness of the membrane 205, or different. For example, the nanopore 204 can span at least about 50%, 60%, 70%, or 80% the thickness of the membrane 205.

The sensor 200 can further include a complexing moiety 209 coupled to the membrane 205. The complexing moiety 209 can be coupled to the membrane 205 through a linker 210. As an alternative, the complexing moiety 209 is coupled to the nanopore 204, such as the lip of the nanopore 204 or a portion of a channel of the nanopore 204 that is directed through the membrane 205. The location of the linker 210 can be selected to provide such coupling.

The membrane 205 includes a trans side and a cis side. The complexing moiety 209 is disposed at the trans side of the membrane 205. The cis side is opposite from the trans side.

The linker 210 can be a molecule that includes one or more nucleic acid or amino acid moieties, such as a polynucleotide or polypeptide. The linker 210 can be a polymer. In some cases, the linker 210 is a polymer such as a peptide, nucleic acid, polyethylene glycol (PEG). The linker 210 can be any suitable length. For example, the linker 210 can have a length that is at least about 5 nm, 10 nm, 30 nm, 40 nm, 50 nm, or 100 nm. The linker 210 can be rigid or flexible.

The complexing moiety 209 can include a coupling domain the permits coupling to the linker 210. Example coupling domains (which can be coupled to the complexing moiety 209, e.g., as an in frame fusion domain or as a chemically coupled domain) include any of an added recombinant dimer domain of the enzyme, a large extraneous polypeptide domain, a polyhistidine tag, a HIS-6 tag, a biotin, an avidin sequence, a GST sequence, a glutathione, a BiTag (AviTag) sequence, an S tag, a SNAP-tag, an antibody, an antibody domain, an antibody fragment, an antigen, a receptor, a receptor domain, a receptor fragment, a ligand, a dye, an acceptor, a quencher, and/or a combination thereof.

As an alternative, the linker 210 can be precluded and the complexing moiety 209 can be directly linked to the membrane 205. The complexing moiety 209 can include a coupling domain that permits direct coupling (e.g., attachment) to the membrane 205. The complexing moiety 209 can be coupled to the membrane 205 or the nanopore 204, such as the lip of the nanopore 204 or a portion of a channel of the nanopore 204 that is directed through the membrane 205. In some cases, the complexing moiety 209 is covalently coupled to the nanopore 204 or membrane 205.

The solution 202 can have an electrolyte. The electrolyte can include one or more salts, such as NaCl, KCl, or AgCl. The solution 202 can have a salt concentration that permits the detection of a current using the first electrode 201 and second electrode 203. In an example, the concentration can be from about 0.1 mole/liter (M) to 10 M, or 2 M to 8 M. As another example, the concentration can be from 0.1 mM to 10 mM, or 0.5 mM to 5 mM.

The solution 202 can include a buffer for PCR. For example, the solution 202 can include 50 mM to 200 mM Tris-HCl (e.g., 100 mM Tris-HCl), 200 mM to 1000 mM KCl (e.g., 500 mM KCl), and 0.5 mM to 5 mM MgCl₂.

The first electrode 201 and second electrode 203 can be formed of one or more metals. In some cases, the first electrode 201 and second electrode 203 are formed of Au, Ag or Pt. For example, the first electrode 201 is formed of Pt and the second electrode 203 is formed of Ag. As an alternative, the first electrode 201 is formed of Pt and the second electrode 203 is formed of AgCl.

In some cases, the second electrode 203 is formed of a material that permits the electrochemical depletion of the electrode 203 during detection. For example, the second electrode 203 can be formed of AgCl. During operation of the sensor 200, AgCl→Ag⁺+Cl⁻. This can be reversed by applying an inverse electrical potential to the second electrode 203 to deposit AgCl onto the second electrode 203, thereby reversing depletion.

In some cases, the sensor 200 is operated by application of a direct current (DC) voltage to the second electrode 203 relative to the first electrode 201. The voltage can range from 0.5 volts (V) to 20 V, or 1 V to 10 V. In such DC operation, the voltage can be reversed (i.e., V→−V→V). As an alternative, the sensor is operated by application of an alternating current (AC) voltage to the second electrode 203 relative to the first electrode 201. The voltage can range from 0.5 V to 20 V, or 1 V to 10 V.

During operation of the sensor 200, an electric field can be provided across the nanopore 204 upon the application of a voltage between the first electrode 201 and the second electrode 203. The electric field can be configured to direct a target nucleic acid molecule in the solution 202 towards the nanopore 204. The electric field can aid the target nucleic acid molecule to approach and move through the nanopore 204. As an alternative or in addition to, a pressure drop can be provided across the nanopore 204, which can aid the target nucleic acid molecule to approach and move through the nanopore 204. In some cases, a pressure-derived force exceeds the opposing voltage-derived force. The motion of the target nucleic acid molecule can be regulated using the combination of a pressure drop and an electric field. For instance, the movement of the target nucleic acid molecule can be slowed upon the application of an electric field from the trans side to the cis side of the membrane 205 while applying a pressure drop from the cis side to the trans side. As an alternative, the movement of the target nucleic acid molecule can be accelerated upon the application of an electric field from the cis side to the trans side of the membrane 205 while applying a pressure drop from the cis side to the trans side.

In some cases, the pressure-derived and voltage-derived forces are balanced to regulate (e.g., increase or decrease) a translocation time (or dwell time) of a target nucleic acid molecule through the nanopore 204. The charge can be deduced from the balance of forces on the molecule via the relationship qE=Fmech, where ‘E’ is the electric field in the nanopore 204, which can be a function of the voltage applied between the electrodes 201 and 203, and Fmech is the sum of the mechanical forces on the target nucleic acid molecule from the applied pressure and/or the fluid flow through the nanopore 204.

In some cases, the solution 202 has a Mg²⁺ concentration that is less than 100 M, 50 M, 40 M, 30 M, 20 M, 10 M, 5 M, 1 M, 0.1 M, 0.01 M, or 0.001 M. A sample provided to the sensor 200 for detection can have a Mg²⁺ concentration that is less than 100 M, 50 M, 40 M, 30 M, 20 M, 10 M, 5 M, 1 M, 0.1 M, 0.01 M, or 0.001 M.

The complexing moiety 209 can be a protein, such as an enzyme. The complexing moiety 209 can be an enzyme other than a polymerase. The complexing moiety 209 can be an enzyme other than a molecular motor. In some examples, the complexing moiety 209 is an endonuclease or exonuclease. The complexing moiety 209 can be an enzyme that has reduced activity or is not enzymatically active. Conditions of the solution can be selected such that the enzyme has reduced activity or is not enzymatically active. The conditions can be selected from the group consisting of salt (or ion) concentration and temperature of the sample.

In some cases, a concentration of an ion (e.g., Mg²⁺) in the solution 202 can be selected such that the enzyme is not enzymatically active. For example, the complexing moiety 209 is an endonuclease and the solution 202 has a Mg²⁺ concentration that is less than about 1 M, 0.1 M, 0.01 M, or 0.001 M. In another example, the complexing moiety 209 is an endonuclease and the solution 202 has a Mg²⁺ concentration that is less than about 1 M, 0.1 M, 0.01 M, or 0.001 M.

As an alternative, the complexing moiety 209 is an enzyme that is enzymatically active. In such a case, a concentration of an ion in the solution 202 can be selected such that the enzyme is enzymatically active.

The complexing moiety 209 can be a protein that binds or otherwise interacts with a target nucleic acid molecule. In some cases, a binding strength associated with such interaction is greater than a binding strength associated with the interaction between the complexing moiety 209 and other molecules, such as nucleic acid molecules other than the target nucleic acid molecule.

In some cases, the complexing moiety 209 has at least one active site or other interaction domain (e.g., binding domain) that is tailored to interact with the target nucleic acid molecule. The active site can be synthesized or generated using molecular evolution. For example, when a DNA or RNA sequence of a target nucleic acid is known, the active site can be evolved using phage display or ribosome display for protein evolution. The protein sequence can be randomly mutagenized and selected for each round, and a group of protein with high binding affinity can be finally evolved after several rounds of evolution.

As an alternative, the complexing moiety 209 is a primer. The primer can have a sequence that is specific to the target nucleic molecule, such as a sequence that is complementary to at least a portion of the target nucleic acid molecule. In some examples, the primer is a universal primer that is configured to hybridize to the target nucleic acid molecule regardless of a sequence of the target nucleic acid molecule.

The complexing moiety 209 can be coupled to the membrane 205 or nanopore 204 by exposing the membrane 205 or nanopore 204 to the complexing moiety 209. As an alternative, the complexing moiety 209 does not couple to the membrane 205 but is present in the solution 202 and couples to the target nucleic acid molecule in the solution 202 to form a complex comprising the target nucleic acid molecule coupled to the complexing moiety 209. Upon the application of an electrical potential across the membrane 205, the target nucleic acid molecule is directed through the nanopore 204 but is stopped or stalled upon interaction between the complex and the membrane 205 or nanopore 204.

During use of the sensor 200, the circuit 206 provides an electrical potential across the first electrode 201 and the second electrode 203. An electrolyte in the solution 202 can transport ions in the solution 202 through the nanopore 204. During use, the second electrode 203 can undergo an oxidation reaction to yield ions of the second electrode 203 in the solution 202, which can be directed through the nanopore towards the first electrode 201. A reduction reaction can occur at the first electrode 201 using ions in the solution 202.

Upon the flow of the solution 202 through the nanopore 204, a measurable current can be detected using the first electrode 201 and the second electrode 203. The current can change with a change in flow rate of a flow through the nanopore 204. For example, upon obstruction of the nanopore 204 with a target nucleic acid molecule, the flow rate can change, which can lead to change in current measured by the first electrode 201 and the second electrode 203. Such change in current can be related to the size and time of the obstruction. A molecule that obstructs the nanopore 204 for a longer period of time can effect a current change for a longer period of time, which can be proportional to the dwell time of the molecule in the nanopore. The intensity of the change in current can be directly related to the size of the obstruction. For example, a larger molecule in or flowing through the nanopore 204 can effect a larger change in current as compared to a smaller molecule in or flowing through the nanopore 204.

The circuit 206 can reverse the direction of the electrical potential across the first electrode 201 and the second electrode 203. This can aid in reversing any depletion of the second electrode 203. For example, to deposit ions from the solution 202 on the second electrode, the electrical potential across the first electrode 201 and the second electrode 203 can be reversed, which can provide a reduction reaction at the second electrode 203 (e.g., Ag⁺+Cl⁻→AgCl).

Nanopore sensors of the present disclosure can be used to detect a target nucleic acid molecule. Such detection can be facilitated by increasing a dwell time of the target nucleic acid molecule in, adjacent, or in proximity to a nanopore of a nanopore sensor, thereby affecting fluid flow through the nanopore. This can generate a measurable current or change in current at electrodes of the nanopore sensor. The dwell time of the target nucleic acid molecule can be increased using a complexing moiety.

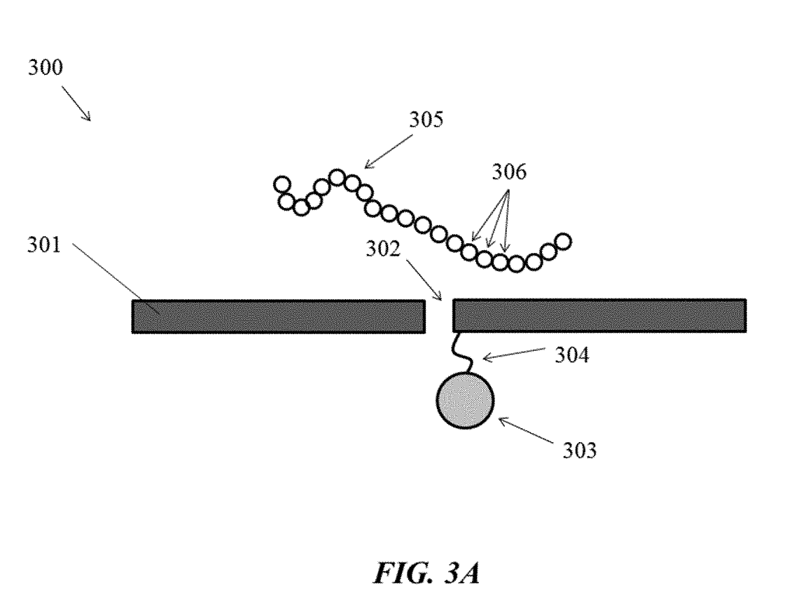

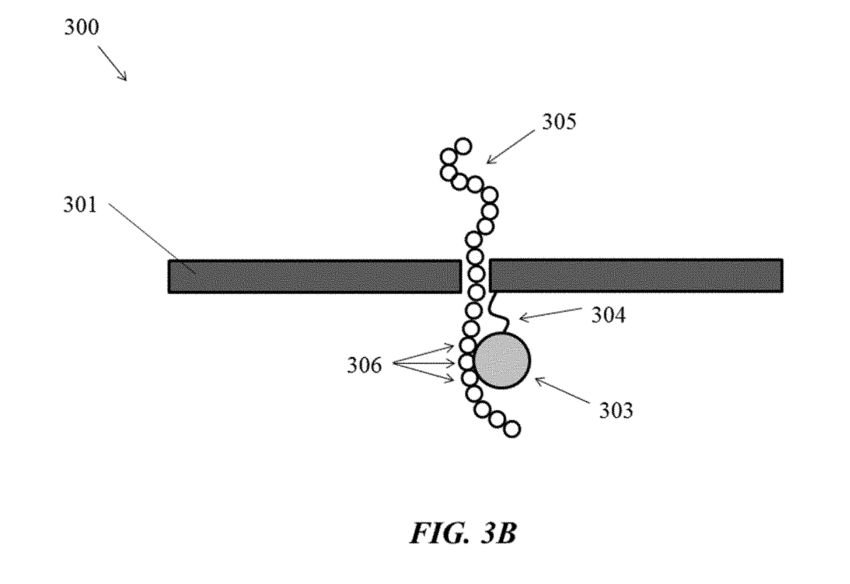

FIGS. 3A-3B schematically illustrate the detection of a target nucleic acid molecule using a nanopore sensor 300. With reference to FIG. 3A, the nanopore sensor 300 includes a membrane 301 having a nanopore 302. A complexing moiety 303 is coupled to the membrane 301 via a linker 304. A target nucleic acid molecule 305 is disposed in proximity to the membrane 301 at a cis side of the membrane 301. The target nucleic acid molecule 305 includes contiguous nucleic acid subunits 306 (or nucleotides). The nanopore sensor 300 includes electrodes (not shown), which can be as described elsewhere herein. The target nucleic acid molecule 305 can be directed to the nanopore 302 upon the application of an electrical potential (V) between the electrodes, which can provide an electric field that directs the target nucleic acid molecule 305 to the nanopore 302. In cases in which the complexing moiety 303 is an enzyme, a solution having the target nucleic acid molecule 305 can have conditions selected such that the enzyme has reduced activity. For example, if the complexing moiety 303 is an exonuclease, the solution can have a Mg²⁺ concentration that is less than about 1 M, 0.1 M, 0.01 M, or 0.001 M.

In FIG. 3B, the target nucleic acid molecule 305 is directed through the nanopore 302, such as upon the application of the electrical potential between the electrodes. The target nucleic acid molecule 305 interacts with the complexing moiety 303 such that a complex is formed between the target nucleic acid molecule 305 and the complexing moiety 303. The complexing moiety 303 can specifically bind to the target nucleic acid molecule 305. Such interaction can slow or stop the flow of the target nucleic acid molecule 305 through the nanopore 302, which increases the dwell time of the target nucleic acid molecule 305 in the nanopore 302. The increased dwell time can be detected by the electrodes as a measureable change in current (C) or current change (dC/dt) with time.

The target nucleic acid molecule 305 can be detected without obtaining a nucleic acid sequence of the target nucleic acid molecule 305 from sequential measurements of the current or change thereof upon the flow of the sample through the nanopore 302. The current or change thereof can be detected at a dwell time that is indicative of the presence of the target nucleic acid molecule 305. For example, a current measured from 1 millisecond (ms) and 10 ms can be indicative of the presence of the target nucleic acid molecule 305, while a current measured at less than 1 ms can be indicative of other molecules or species in solution that may not be target nucleic acid molecule 305.

The target nucleic acid molecule 305 can be detected by measuring a current or change thereof upon the flow of a sample having or suspected of having the target nucleic acid molecule 305 through the nanopore 302. The measured current or change thereof can be compared to a reference (e.g., baseline current or current change). Any difference with respect to such reference as a function of time can be indicative of the presence of the target nucleic acid molecule 305.

Subsequent to detecting the target nucleic acid molecule 305, a stimulus can be provided to remove the target nucleic acid molecule 305 from the nanopore 302. The stimulus can be a pressure pulse, heat pulse, voltage pulse, the application of a sheer force, or a combination thereof. The stimulus can break the interaction between the complexing moiety 303 and the target nucleic acid molecule 305. In an example, the interaction between the complex and the nanopore imparts a shear force to the complexing moiety 303 that disrupts the interaction between the complexing moiety 303 and the target nucleic acid molecule 305. This can disrupt the complex and permit the target nucleic acid molecule to exit the nanopore 302.

In some examples, the stimulus is a voltage pulse supplied by between the electrodes of the nanopore sensor. The voltage pulse can include a voltage from about 0.5 V to 20 V, or 1 V to 10 V, and supplied for at time period from about 500 nanoseconds (ns) and 2 ms, or 500 ns and 1 ms. For example, the voltage pulse is an electrical potential of 5 V for a time period of about 1 ms. In some cases, the pulse duration is less than or equal to about 5 ms, 4 ms, 3 ms, 2 ms, or 1 ms.

The stimulus can be applied to the membrane 301 and/or the nanopore 302. The stimulus can be directed to the membrane 301 and/or the nanopore 302 under conditions such that the membrane 301 and/or the nanopore 302 is not disrupted.

Upon application of a stimulus, the target nucleic acid molecule 305 can flow out of the nanopore 302 from the cis side to the trans side of the membrane 301. Once removed from the nanopore 302, the nanopore 302 and the complexing moiety 303 can be used to detect the presence of another target nucleic acid molecule in solution.

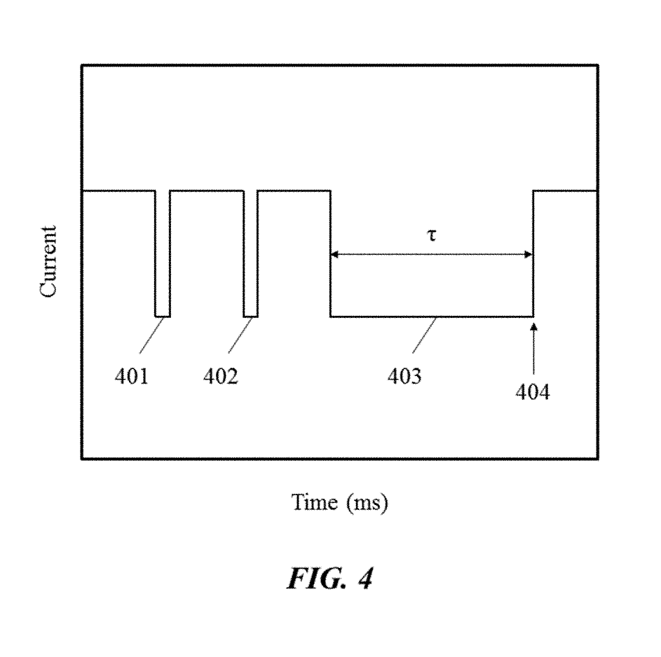

FIG. 4 shows an example plot of current measurement (y axis) with time (x axis, milliseconds (ms)) using a nanopore sensor of the present disclosure. The nanopore sensor includes a membrane with a nanopore. Over the detection time period, the flow of a solution through the nanopore is slowed or otherwise disrupted three times, yielding current signals 401, 402 and 403. Each change in current 401-403 has a dwell time (t). Comparing the dwell time to a reference can lead to the determination that the current signal 403 is associated with a target nucleic acid molecule and the signals 401 and 402 are not associated with the target nucleic acid molecule. The determination can be made by measuring a change in current (e.g., ΔC versus time or dC/dt versus time) that is indicative of the presence of the complex. For example, from reference measurements (i.e., with a sample having a known target nucleic acid molecule), any dwell time greater than or equal to 5 ms can be attributed to a target nucleic acid molecule. The signals 401 and 402 have dwell times of about 1 ms, and the signal 403 has a dwell time that is greater than 5 ms.

The signal 403 can persist until a stimulus is applied to the nanopore and/or the membrane to remove the target nucleic acid molecule from the nanopore. In the illustrated example, a voltage pulse is applied to the nanopore and/or the membrane at time 404.

The signals 401 and 402 that are not associated with the target nucleic acid molecule can each persist for a given period of time independent of the stimulus. The signal 403 can persist until the stimulus is applied at time 404.

The amplitude of the signals 401, 402 and 403 can be the same or different. In some cases, the amplitude of the signal 403 is different than the amplitude of the signals 401 and 402.

The nanopore sensor can measure current continuously or periodically. In some cases, the nanopore sensor measures current subsequent to facilitating the flow of a solution with a sample having or suspected of having the target nucleic acid molecule through the nanopore.

### Methods for Forming Nanopores

Nanopores of the present disclosure can be formed via a variety of methods. For instance, an array of one or more nanopores can be formed using photolithography in which a pattern of one or more holes is defined in a photoresist (e.g., poly(methyl methacrylate)) and transferred to a substrate (e.g., silicon substrate) using photolithography, which can include exposing the pattern of one or more holes to an anisotropic chemical etchant.

In some cases, a substrate is provided and a photoresist layer is provided adjacent to the substrate. The photoresist layer can be formed of, for example, poly(methyl methacrylate) (PMMA), poly(methyl glutarimide) (PMGI), phenol formaldehyde resin, or an expoxy-based negative photoresist (e.g., SU-8). The photoresist can be developed upon exposure to light, such as ultraviolet (UV) light

Next, the photoresist can be exposed to a pattern of electromagnetic radiation or particles (e.g., light or electron beam) to define a hole in the photoresist that exposes the substrate. The exposure to light can cause a chemical change that allows some of the photoresist to be removed by a wash solution, leaving the hole. A positive photoresist can become soluble in the wash solution when exposed, while in a negative photoresist, unexposed regions are soluble in the wash solution. Next, the hole can be exposed to a chemical etchant. The chemical etchant can provide anisotropic etching. For example, the chemical etchant can be potassium hydroxide (KOH). In some cases, a focused ion beam and/or a time buffered oxide etch (BOE) can be used to provide fine etching, such as removal of residual oxide.

The substrate can be a semiconductor or polymer substrate. For example, the substrate can be formed of silicon, germanium, carbon (e.g., graphene), or gallium arsenide, or an oxide or nitride thereof. As an example, the substrate is formed of silicon, silicon oxide or silicon nitride. As another example, the substrate can be formed of a metal, such as copper, nickel, or aluminum. The substrate can have a thickness from about 10 nm to 5000 nm, or 20 nm to 1000 nm, or 30 nm to 1000 nm. In an example, the substrate has a thickness from about 50 nm to 150 nm.

Nanopores formed according to methods provided herein can have various electrical conductances. For example, a nanopore having a cross-sectional size from about 5 nm to 15 nm can have an electrical conductance from about 20 nano Siemens (nS) to 150 nS, 50 nS to 120 nS, or 60 nS and 110 nS. Such conductance may be measured with respect to the flow of an electrolyte, such as KCl.

### Computer Control Systems

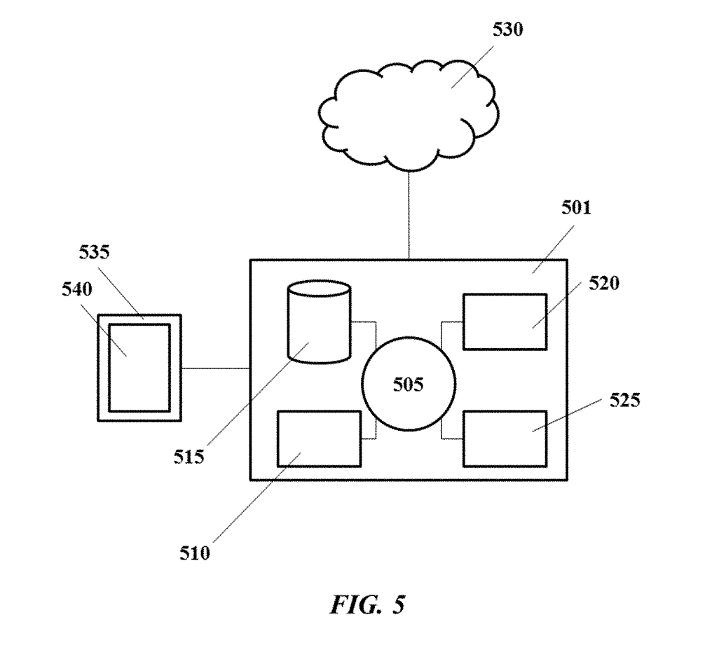

The present disclosure provides computer control systems that are programmed to implement methods of the disclosure. FIG. 5 shows a computer system 501 that is programmed or otherwise configured to detect the presence of a target nucleic acid sample in solution. The computer system 501 can regulate various aspects of nanopore sensor of the present disclosure, such as, for example, detecting current or current change with time. The computer system 501 can be in communication with a nanopore sensor, which can be part of a chip. The computer system 501 can be stationary or mobile. In some examples, the computer system 501 is part of a mobile electronic device.

The computer system 501 includes a central processing unit (CPU, also “processor” and “computer processor” herein) 505, which can be a single core or multi core processor, or a plurality of processors for parallel processing. The computer system 501 also includes memory or memory location 510 (e.g., random-access memory, read-only memory, flash memory), electronic storage unit 515 (e.g., hard disk), communication interface 520 (e.g., network adapter) for communicating with one or more other systems, and peripheral devices 525, such as cache, other memory, data storage and/or electronic display adapters. The memory 510, storage unit 515, interface 520 and peripheral devices 525 are in communication with the CPU 505 through a communication bus (solid lines), such as a motherboard. The storage unit 515 can be a data storage unit (or data repository) for storing data. The computer system 501 can be operatively coupled to a computer network (“network”) 530 with the aid of the communication interface 520. The network 530 can be the Internet, an internet and/or extranet, or an intranet and/or extranet that is in communication with the Internet. The network 530 in some cases is a telecommunication and/or data network. The network 530 can include one or more computer servers, which can enable distributed computing, such as cloud computing. The network 530, in some cases with the aid of the computer system 501, can implement a peer-to-peer network, which may enable devices coupled to the computer system 501 to behave as a client or a server.

The CPU 505 can execute a sequence of machine-readable instructions, which can be embodied in a program or software. The instructions may be stored in a memory location, such as the memory 510. The instructions can be directed to the CPU 505, which can subsequently program or otherwise configure the CPU 505 to implement methods of the present disclosure. Examples of operations performed by the CPU 505 can include fetch, decode, execute, and writeback.

The CPU 505 can be part of a circuit, such as an integrated circuit. One or more other components of the system 501 can be included in the circuit. In some cases, the circuit is an application specific integrated circuit (ASIC).

The storage unit 515 can store files, such as drivers, libraries and saved programs. The storage unit 515 can store user data, e.g., user preferences and user programs. The computer system 501 in some cases can include one or more additional data storage units that are external to the computer system 501, such as located on a remote server that is in communication with the computer system 501 through an intranet or the Internet.

The computer system 501 can communicate with one or more remote computer systems through the network 530. For instance, the computer system 501 can communicate with a remote computer system of a user (e.g., service provider). Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 501 via the network 530.

Methods as described herein can be implemented by way of machine (e.g., computer processor) executable code stored on an electronic storage location of the computer system 501, such as, for example, on the memory 510 or electronic storage unit 515. The machine executable or machine readable code can be provided in the form of software. During use, the code can be executed by the processor 505. In some cases, the code can be retrieved from the storage unit 515 and stored on the memory 510 for ready access by the processor 505. In some situations, the electronic storage unit 515 can be precluded, and machine-executable instructions are stored on memory 510.

The code can be pre-compiled and configured for use with a machine have a processer adapted to execute the code, or can be compiled during runtime. The code can be supplied in a programming language that can be selected to enable the code to execute in a pre-compiled or as-compiled fashion.

Aspects of the systems and methods provided herein, such as the computer system 501, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

A machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 501 can include or be in communication with an electronic display 535 that comprises a user interface (UI) 540 for providing, for example, signals from a nanopore sensor with time. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 505.

### EXAMPLE 1

A semiconductor substrate (e.g., silicon) is irradiated with energetic particles in a processing chamber. The energetic particles can be argon ions (e.g., Ar+). At least one nanopore is generated in the semiconductor substrate using photolithography and etching. For example, a mask can be provided adjacent to the semiconductor and locations of the mask corresponding to the nanopore is exposed and the mask in such locations is removed to expose a portion of the semiconductor substrate. The exposed portion of the semiconductor substrate is contacted with an etching solution (e.g., mixture of HF and HNO₃) to etch the nanopore into the semiconductor substrate. An etch block layer in the semiconductor substrate can terminate the etching. The semiconductor substrate with nanopore can be provided adjacent to electrodes to provide the nanopore sensor.

The nanopore sensor can be provided in an effendorf PCR tube, including chamber for PCR reaction and for detection. The semiconductor with the nanopore can be a membrane that separates two wells, a cis well and a trans well. Reagents used for nucleic acid amplification (e.g., isothermal amplification) are added to the cis well. Reagents for nucleic acid amplification can include a PCR buffer, primer, DNA polymerase, template nucleic acid sample, and primers with the restriction sites in the 5′ end. LAMP and endonuclease can be conducted at a temperature of about 65° C. to generate a double stranded target nucleic acid molecule as an amplification product of the template nucleic acid molecule. Next, the endonuclease is covalently cross-linked with the nanopore. A voltage is applied between the cis and trans wells and a current is measured with the nanopore sensor.

The voltage between the wells (across the cis and trans sides) induces the negatively charged target nucleic acid molecule to enter and electrophorese through the nanopore. The target nucleic acid molecule has an endonuclease target sequence that binds to the endonuclease to form a complex that increases the dwell time of the target nucleic acid molecule in the nanopore. The complex is stable until it dissociates, such as by the sheer force of the nanopore. Based on the increased dwell time, the presence of the target nucleic acid molecule is identified.

### EXAMPLE 2

A thin film of 2 μm wet thermal silicon oxide and 100 nm low pressure chemical vapor deposition (LPCVD) low-stress (silicon rich) silicon nitride are deposited on 500 μm thick P-doped (100) Si wafers of 1-20 ohm·cm resistivity. Freestanding 20 μm membranes are formed by anisotropic KOH (33%, 80° C.) etching of wafers in which the thin films has been removed in a photolithographically patterned region by reactive ion etching. A focused ion beam (Micrion 9500) is used to remove about 1.5 μm of silicon oxide in a 1 μm square area in the center of the freestanding membrane. A subsequent BOE removes about 600 nm of the remaining oxide, leaving a 2 μm free-standing mini-membrane of silicon nitride in the center of the freestanding oxide/nitride membrane. The nitride film is about 80 nm thick after processing in KOH and BOE, as measured by ellipsometry and cross-sectional transmission electron microscopy (TEM). A focused 200 keV electron beam from a JEOL 2010F field-emission TEM (JEOL USA, Peabody, Mass.) is used to form roughly hourglass-shaped nanopores in the center of the nitride minimembrane. Nanopore diameters are about 10 nm.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

1. A method for assaying the presence of a target nucleic acid molecule in a sample having or suspected of having said target nucleic acid molecule, said target nucleic acid molecule being coupled to a complexing moiety, the method comprising:
(a) facilitating the flow of said sample through at least one nanopore in a membrane disposed adjacent or in proximity to an electrode that is adapted to detect a current or change thereof upon movement of a complex having said target nucleic acid molecule coupled to said complexing moiety through said at least one nanopore, wherein said movement takes a dwell time that is longer than that of the movement of said target nucleic acid molecule through said at least one nanopore when said target nucleic acid molecule is not coupled to said complexing moiety; and
(b) measuring said current or change thereof with said electrode upon facilitating the flow of said sample through said at least one nanopore; and
(c) detecting said complex in said sample from said current or change thereof measured in (b) without obtaining a nucleic acid sequence of said target nucleic acid molecule, thereby assaying the presence of said target nucleic acid molecule in said sample.

2. The method of claim 1, wherein said complexing moiety is coupled to said membrane.

3. (canceled)

4. The method of claim 1, wherein said complexing moiety is coupled to said nanopore.

5. (canceled)

6. The method of claim 1, wherein said complexing moiety is a protein or is a primer.

7. (canceled)

8. The method of claim 6, wherein said protein binds to said target nucleic acid molecule at a binding strength that is greater than a binding strength for any other nucleic acid molecule.

9-11. (canceled)

12. The method of claim 1, wherein said sample has a Mg²⁺ concentration that is less than 1 mole/liter (M).

13-15. (canceled)

16. The method of claim 1, further comprising, prior to (a), (i) providing a reaction mixture including a biological sample having or suspected of having a template nucleic acid molecule as a precursor of said target nucleic acid molecule, at least one primer that is complementary to said template nucleic acid molecule, and a polymerase, and (ii) subjecting said reaction mixture to a nucleic acid amplification reaction under conditions that yield said target nucleic acid molecule in said sample.

17-19. (canceled)

20. The method of claim 16, wherein said primer has one or more restriction sites or binding sites for said complexing moiety.

21. (canceled)

22. The method of claim 16, wherein said nucleic acid amplification reaction is isothermal amplification.

23-27. (canceled)

28. The method of claim 1, wherein said current is measured subsequent to facilitating the flow of said sample through said at least one nanopore.

29. (canceled)

30. The method of claim 1, wherein said at least one nanopore has a cross-sectional size that is from about 0.5 nanometer (nm) to 30 nm.

31. (canceled)

32. (canceled)

33. The method of claim 1, wherein said membrane is a lipid bilayer or a solid state membrane.

34-37. (canceled)

38. The method of claim 1, wherein said at least one nanopore is a pore-forming protein in said membrane.

39. (canceled)

40. The method of claim 1, wherein said facilitating comprises applying an electrical potential across said at least one nanopore.

41. (canceled)

42. The method of claim 40, wherein said electrical potential is from about 1 V to 10 V relative to a reference.

43. The method of claim 1, further comprising applying a pulse of an electrical potential across said at least one nanopore to decouple said complexing moiety from said target nucleic acid molecule, which pulse is applied subsequent to facilitating the flow of said sample through said at least one nanopore.

44. The method of claim 1, wherein said at least one nanopore is adjacent or in proximity to an additional electrode.

45. (canceled)

46. The method of claim 1, wherein said complexing moiety increases said dwell time upon interaction of said complexing moiety with said at least one nanopore.

47. The method of claim 1, wherein said at least one nanopore includes a plurality of nanopores.

48. (canceled)

49. The method of claim 1, wherein said target nucleic acid molecule is detected without obtaining a nucleic acid sequence of said target nucleic acid molecule from sequential measurements of said current or change thereof upon the flow of said sample through said at least one nanopore.

50. The method of claim 1, wherein said current or change thereof is detected at a dwell time that is indicative of the presence of said target nucleic acid molecule.

51. The method of claim 1, wherein said target nucleic acid molecule includes at least 5 contiguous nucleotide bases.

52-57. (canceled)

58. A method for assaying the presence of a target nucleic acid molecule in a sample having or suspected of having said target nucleic acid molecule, said target nucleic acid molecule being coupled to a protein other than a polymerase, the method comprising:
(a) facilitating the flow of said sample through at least one nanopore in a membrane disposed adjacent or in proximity to an electrode that is adapted to detect a current or change thereof upon movement of a complex having said target nucleic acid molecule coupled to said protein through said at least one nanopore, wherein said movement takes a dwell time that is longer than that of the movement of said target nucleic acid molecule through said at least one nanopore when said target nucleic acid molecule is not coupled to said protein;
(b) measuring said current or change thereof with said electrode upon facilitating the flow of said sample through said at least one nanopore; and
(c) detecting said complex in said sample from said current or change thereof measured in (b), thereby assaying the presence of said target nucleic acid molecule in said sample.

59. A method for assaying the presence of a target nucleic acid molecule in a sample having or suspected of having said target nucleic acid molecule, said target nucleic acid molecule being coupled to an enzyme under conditions such that the enzyme is not enzymatically active, the method comprising:
(a) facilitating the flow of said sample through at least one nanopore in a membrane disposed adjacent or in proximity to an electrode that is adapted to detect a current or change thereof upon movement of a complex having said target nucleic acid molecule coupled to said enzyme through said at least one nanopore, wherein said movement takes a dwell time that is longer than that of the movement of said target nucleic acid molecule through said at least one nanopore when said target nucleic acid molecule is not coupled to said enzyme;
(b) measuring said current or change thereof with said electrode upon facilitating the flow of said sample through said at least one nanopore; and
(c) detecting said complex in said sample from said current or change thereof measured in (b), thereby assaying the presence of said target nucleic acid molecule in said sample.

60. The method of claim 59, wherein said conditions are selected from the group consisting of salt concentration of said sample and temperature of said sample.

61. The method of claim 60, wherein said salt concentration includes a concentration of Mg²⁺.

62. The method of claim 61, wherein said concentration is less than 1 mole/liter (M).

63-86. (canceled)
